(19) United States
(12) Patent Application Publication
(10) Pub. No.: US 2006/0178281 A1
(43) Pub. Date: Aug. 10, 2006

(54) Food-grade sanitizing composition

(76) Inventor: Richard Alasri, Halluin (FR)

Correspondence Address: HOVEY WILLIAMS LLP, 2405 GRAND BLVD., SUITE 400, KANSAS CITY, MO, 64108, US

(21) Appl. No.: 10/566,543

(22) PCT Filed: Aug. 4, 2003
(86) PCT No.: PCT/SE03/01248

§ 371 Date: Jan. 31, 2006

(51) Int. Cl. C11D 3/00

(52) U.S. Cl. 510/160

(57) ABSTRACT

A sanitizing composition of food-grade quality is provided which is capable of being loaded onto an absorbant substrate to form a sanitizing wipe. Inventive compositions include a sulfur-containing salt, a carboxylic acid or salt thereof, and an alcohol and may be used to disinfect hard surfaces and animal skin, particularly bovine teats.

### BACKGROUND OF THE INVENTION

1. Field of the Invention

The present invention is directed toward a sanitizing composition which is based on natural and food-grade materials. The inventive composition is useful for cleaning food contact surfaces and animal skin, particularly bovine teats. The composition may also be loaded onto woven or unwoven towels so as to provide ready-to-use moist towels for general and hygienic cleaning.

2. Description of the Prior Art

Bacteria and microbe control has been an ongoing issue for the agricultural and food preparation industry. A number of germicidal compounds are presently available which are highly effective in controlling undesired bacteria and microbes. Such compositions include iodine and chlorine-based disinfecting agents. However, once a surface or area has been disinfected, care must be exercised so that the antimicrobial composition does not contaminate the food product Generally, this has required the additional step of rinsing the surface with water so as to wash away the disinfecting agent.

In the dairy industry, preparation of a cow for milking can be a very labor intensive and time consuming part of the milking procedure. With conventional antimicrobial compounds, the compound is first applied to the udder and teat area by spraying or dipping, and then the udder is dried with an absorbent towel. Care must be taken to ensure that the cleansing composition has been removed from the udder and teat prior to milking so that the milk supply does not become contaminated. After milking, the teat area is also disinfected to guard against the dairy cow developing mastitis.

A similar microbe control problem exists in the restaurant and food preparation industry. Food preparation areas, especially those coming into contact with uncooked meat, require thorough cleaning and disinfecting to prevent the growth and spread of harmful bacteria. However, as in the dairy industry, care must be taken to ensure that the harsh disinfectant product is rinsed from the food preparation area to avoid the food from becoming contaminated with the disinfecting agent. This adds additional time and labor to the cleaning process.

A number of composition and premoistened towel products have been proposed to in response to this problem For example, WO 96/39842 discloses a disinfectant composition which can be applied to a paper or fabric towel to form a disinfectant wipe. The disinfectant composition employs a bacteriocin (such as lanthocin, nisin, cinnamycin, and epidermin) which is a powerful antibiotic compound as the disinfecting agent The wipes can be used for surface cleaning and disinfecting of cow teats.

WO 02/40628 describes a premoistened wipe for use on food preparation surfaces. The wipe includes a substrate and a composition comprising at least one of a toxicologically-acceptable anionic surfactant, chelant, nonioninc surfactant, buffer, preservative, suds suppressor, perfume, and aqueous carrier. However, this composition has not been shown to be effective in controlling the bacteria which cause mastitis.

Therefore, there is a real and unfulfilled need for a sanitizing composition which is of a food-grade quality that will effectively control the growth and spread of disease causing microbes without global contamination of food products which come into contact with the composition so as to render the products unfit for human consumption

### SUMMARY OF THE INVENTION

The present invention overcomes the above problems and provides a food-grade sanitizing composition comprising a sulfur-containing salt (preferably an inorganic sulfur-containing salt), a substituted or unsubstituted C2-C10 carboxylic acid (preferably a monocarboxylic acid) or salt thereof, and an alcohol. As used herein, the term “sulfur-containing salt” refers to any salt which comprises at least one sulfur atom, regardless of whether the sulfur atom is itself covalently bound to an additional non-metal species. Furthermore, as used herein, the term “alcohol” generally refers to any compound having an alcohol moiety and includes diols, triols, and polyols. Preferably, the sulfur-containing salt is selected from the group consisting of alkali and alkaline earth metal sulfate and sulfite salts, with sodium sulfate being particularly preferred. Preferred carboxylic acids (and corresponding salts) include lactic acid and salts thereof. Preferred alcohols are selected from the group consisting of C2-C8 alcohols and mixtures thereof, with ethanol propanol (including both 1-propanol and 2-propanol), and mixtures thereof being more preferred.

The sanitizing composition generally comprises from about 0.25-10% by weight of the sulfur containing salt, more preferably from about 0.5-5% by weight, and most preferably from about 0.5-1.0% by weight The composition comprises from about 0.25-10% by weight of the carboxylic acid, more preferably from about 0.5-5% by weight, and most preferably 1.0-3.0% by weight. The composition comprises 0.1-30% by weight alcohol, more preferably from about 1-15% by weight, and most preferably from about 1-10% by weight.

Because compositions according to the present invention are of food-grade quality, they may be applied to animal skin, and particularly bovine teats before and after milking. Various emollients, surfactants, thickeners, and skin conditioning agents may be added to the composition in order to make application to the animal skin easier and less harsh. Preferred emollients are selected from the group consisting of glycerin, lanolin, sorbitol, the alkylene glycols (e.g. ethylene glycoland propylene glycol), the polyols, fatty acid esters of polyhydroxylated compounds, and mixtures thereof. Preferred surfactants are selected from the group consisting of nonionic surfactants, anionic surfactants, cationic surfactants, amphoteric surfactants, polyoxyethylene alcohols, polyoxyethylene nonylphenols, polyalkylene oxide block copolymers, polyvinylpyrrolidone, and mixtures thereof. Xanthan gum is a preferred thickener and allantoin is a preferred skin conditioning agent.

The composition may also be used in conjunction with an absorbent substrate to form a sanitizing wipe. The absorbent substrate comprises a woven or nonwoven web of natural fibers, synthetic fibers, or mixtures of natural and synthetic fibers. Preferred natural fiber materials include cellulosic fibers such as wood pulp, cotton, and rayon, while preferred synthetic fibers include polyester and polypropylene. The inventive composition is loaded onto the absorbent substrate which is then packaged in such a manner to avoid volatilization of the sanitizing composition.

The sanitizing composition and wipes may be used for hygienic cleansing of various surfaces, particularly surfaces which may come in contact with food items. The composition exhibits antimicrobial properties which inhibit the spread and growth of microbes. In addition to use on hard surfaces, the composition may be applied to living skin tissue, such as human hands and bovine teats. Because the present inventive composition is of a food-grade quality, it may be applied to bovine teats both before and after milking. Advantageously, the composition allows soil and other potential contaminants to be removed from the teat prior to milking without requiring additional washing to remove the sanitizing composition, and the composition also provides effective protection against mastitis when applied after milking. Should a food product come into contact with the sanitizing composition, the product will not become contaminated itself in such a manner so as to render it unfit for human consumption.

Sanitizing wipes having a quantity of sanitizing composition loaded thereon allow for efficient cleansing of the desired surface. The user need only wipe the surface with the absorbent towel containing the composition in order to provide effective disinfection. No additional rinsing to remove excess sanitizing composition is required prior to surface contact with a food product. The inventive composition also provides efficient preservation of the moist sanitizing wipes during storage. Preferably, the composition preserves the sanitizing properties of the wipes for at least about 6 months over a wide range of temperatures.

### DETAILED DESCRIPTION OF THE PREFERRED EMBODIMENT

The following examples set forth sanitizing composition in accordance with the invention It is to be understood, however, that these examples are provided by way of illustration and nothing therein should be taken as a limitation upon the overall scope of the invention.

### Example 1

Table 1 sets forth comparative and preferred compositions in accordance with the invention All percentages expressed therein are weight percentages unless otherwise stated. Also, it is assumed in each formulation that there is sufficient water to give 100%.

TABLE 1

| Ingredients | | Formulation | | | | | | | | |
|---|---|---|---|---|---|---|---|---|---|---|
| Trade name | Chemical name | #1 | #2 | #3 | #4 | #5 | #6 | #7 | #8 | #9 |
| Amide KDO | Fatty acid diethanolamide | 1% | — | — | 1% | 1% | — | 1% | 1% | 1% |
| Methocell | Hydroxypropylmethyl- | 0.30% | — | 0.30% | 0.30% | — | — | 0.15% | — | — |
| | cellulose | | | | | | | | | |
| Lutensol | Fatty alcohol ethoxylate | 2% | 3% | 2% | 2% | 2% | 2% | 2% | 1.25% | 1.25% |
| Tween 80 | Ethoxylated sorbitan | 2% | 1% | 1% | 2% | 2% | 1% | 2% | 1% | 1% |
| | monooleate | | | | | | | | | |
| | Glycerin | 1% | 1% | 1% | 1% | 1% | 1% | 1% | 1% | 1% |
| Allantoin | 5-ureido-hydantoin | — | — | 0.10% | — | — | 0.10% | — | 0.10% | 0.10% |
| Propylene glycol | 1,2-propanediol | 1% | 1.5% | 1% | 1% | 1% | 1% | 1% | 0.50% | 0.50% |
| | Ethanol | 3.65% | 2.92% | 2.92% | 3.65% | 3.65% | 2.92% | 3.65% | 4.38% | 4.38% |
| | Propanol | 1.35% | 1.08% | 1.08% | 1.35% | 1.35% | 1.08% | 1.35% | 1.62% | 1.62% |
| | Acetic acid | — | — | 1% | — | — | 1% | — | — | — |
| | Hydrogen peroxide | — | — | 0.25% | — | — | 0.25% | — | — | — |
| | Lactic acid | 3% | — | — | 3% | 3% | — | 3% | 2% | 2% |
| | Sodium sulfite | 1% | 3% | 0.50% | 1% | 1% | 0.5% | 1% | — | 0.5% |
| DDBSA | Dodecyl benzenesulfonic acid | — | — | — | — | 2% | — | — | — | — |

The formulations were then tested for their fungistatic capabilities against Aspergillus niger (A. niger). It was observed that formula 2 (without lactic acid) was not effective in vitro against A niger. Also, formula 8 (without sodium sulfite) was not effective against A niger. However, formulation 9 comprising both lactic acid and sodium sulfite exhibited excellent fungistatic properties.

In addition, the minimum inhibitory concentrations for formulas 2, 8, and 9 were determined for Staphylococcus aureus (S. aureus), Pseudomonas aeruginosa (P. aeruginosa), and Escheria coli (E. coli). The results are listed in Table 2. Note that the concentrations are given as a percentage of the sample as prepared in Table 1. For example, an MIC of 20% for formula 2 means a 20% concentration (volume basis) of formula 2 as prepared in Table 1. A “>” means that the composition concentration was beyond the limits of the test.

TABLE 2

| Bacteria | Formula #2 | Formula #8 | Formula #9 |
|---|---|---|---|
| S. aureus | 20.0% | 40.0% | 10.0% |
| P. aeruginosa | >50.0% | >50.0% | 20.0% |
| E. coli | 50.0% | >50.0% | 20.0% |

This test confirms that the compositions using sodium sulfite alone (#2) and lactic acid alone (#8) do not inhibit bacteria growth nearly as well as the composition comprising both lactic acid and sodium sulfite (#9).

### Example 2

This example describes an exemplary sanitizing formulation according to the invention and a method of preparing the same.

In a cleaning vessel 10.0 g of Amide KDO (fatty acid diethanolamine) was added to 756.5 g of demineralized water. The mixture was stirred until the Amide KDO was completely dispersed and the dispersion appeared hazy. Next, 12.5 g of Lutensol AO 109 was added and mixed until completely dissolved. At this time, the mixture appeared clear.

About 5.0 g of sodium sulfite was added to the mixture and stirred until completely dissolved. Then, 10.0 g of Tween 80 (ethoxylated sorbitan monooleate) was added and mixed until completely dissolved While continuing to mix, 10.0 g of glycerin was added, followed by 5.0 g of monopropylene glycol. About 1.0 g of allantoin was added and mixed until completely dissolved. Next, 153.2 g of a mixture of ethanol and isopropanol (27.85% by weight ethanol, 11.35% by weight isopropanol, the balance being water) was added as mixing continued. Twenty (20.0) grams of lactic acid (80%) was added, and the pH of the mixture adjusted to between 5.0 and 5.5 by the addition of 16.8 g of sodium hydroxide (29%). The mixture was then sired for about 30 minutes, or until the pH stabilized.

The composition exhibited stability over a six-month period under accelerated and normal conditions. When applied to paper towels, the composition helped preserve the antimicrobial properties of the wipe for more than six months at both 25° C. and 40° C.

1. A sanitizing composition comprising from about 0.25-10% by weight of a sulfur-containing salt, from about 0.25-10% by weight of a substituted or unsubstituted C2-C10 monocarboxylic acid or a salt thereof, and from about 0.1-30% by weight of an alcohol, said sulfur-containing salt being selected from the group consisting of alkali and alkaline earth metal sulfite salts.

2. The composition of claim 1, said sulfur-containing salt being sodium sulfite.

3. The composition of claim 1, said alcohol being selected from the group consisting of C1-C8 alcohols and mixtures thereof.

4. The composition of claim 3, said alcohol being selected from the group consisting of ethanol, propanol, and mixtures thereof.

5. The composition of claim 1, said composition comprising from about 0.5-5.0% by weight of said sulfur-containing salt.

6. The composition of claim 1, said carboxylic acid comprising lactic acid or a salt thereof.

7. The composition of claim 1, said composition comprising from about 0.5-5.0% by weight of said carboxylic acid or salt thereof.

8. The composition of claim 1, said composition comprising from about 1-15% by weight of said alcohol.

9. The composition of claim 1, said composition further comprising an emollient.

10. The composition of claim 9, said emollient selected from the group consisting of glycerin, lanolin, sorbitol, the alkylene glycols, the polyols, fatty acid esters of polyhydroxylated compounds, and mixtures thereof.

11. The composition of claim 1, said composition further comprising a surfactant.

12. The composition of claim 11, said surfactant being selected from the group consisting of nonionic surfactants, anionic surfactants, cationic surfactants, amphoteric surfactants, polyoxyethylene alcohols, polyoxyethylene nonylphenols, polyalkylene oxide block copolymers, polyvinylpyrrolidone, and mixtures thereof.

13. A sanitizing composition comprising from about 0.25-10% by weight of an inorganic sulfur-containing salt, from about 0.25-10% by weight of a substituted or unsubstituted C2-C10 carboxylic acid or a salt thereof and from about 0.1-30% by weight of an alcohol, said sulfur-containing salt being selected from the group consisting of alkali and alkaline earth metal sulfite salts.

14. The composition of claim 13, said sulfur-containing salt being sodium sulfite.

15. The composition of claim 13, said alcohol being selected from the group consisting of Cl-CS alcohols and mixtures thereof.

16. The composition of claim 15, said alcohol being selected from the group consisting of ethanol, propanol, and mixtures thereof.

17. The composition claim 13, said composition comprising from about 0.5-5.0% by weight of said sulfur-containing salt.

18. The composition of claim 13, said carboxylic acid comprising lactic acid or a salt thereof.

19. The composition of claim 13, said composition comprising from about 0.5-5.0% by weight of said carboxylic acid or salt thereof.

20. The composition of claim 13, said composition comprising from about 1-15% by weight of said alcohol.

21. The composition of claim 13, said composition further comprising an emollient.

22. The composition of claim 21, said emollient selected from the group consisting of glycerin, lanolin, sorbitol, the alkylene glycols, the polyols, fatty acid esters of polyhydroxylated compounds, and mixtures thereof.

23. The composition of claim 13, said composition further comprising a surfactant.

24. The composition of claim 23, said surfactant being selected from the group consisting of nonionic surfactants, anionic surfactants, cationic surfactants, amphoteric surfactants, polyoxyethylene alcohols, polyoxyethylene nonylphenols, polyalkylene oxide block copolymers, polyvinylpyrrolidone, and mixtures thereof.

25. A sanitizing wipe comprising an absorbent substrate and a sanitizing composition according claim 1 loaded thereon.

26. The sanitizing wipe of claim 25, said absorbent substrate comprising a woven or nonwoven web of natural fibers, synthetic fibers, or mixtures of natural and synthetic fibers.

27. The sanitizing wipe of claim 26, said absorbent substrate comprising cellulosic fibers.

28. A method of sanitizing a surface comprising contacting said surface with the composition of claim 1.

29. The method of claim 28, said surface comprising animal skin.

30. The method of claim 29, said surface being a bovine teat.

31. A method of sanitizing a surface comprising wiping said surface with the sanitizing wipe of claim 25.

32. The method of claim 31, said surface comprising animal skin.

33. The method of claim 32, said surface being a bovine teat.
